(19) United States
(12) Patent Application Publication
(10) Pub. No.: US 2010/0198105 A1
(43) Pub. Date: Aug. 5, 2010

(54) NON INVASIVE METHOD FOR DETECTING AN ELECTRONIC PARAMETER DEPENDING ON THE INTRALABYRINTH PRESSURE (PIL) IN A SUBJECT

(76) Inventors: Paul Alexandre Louis Avan, Chamalieres (FR); Thierry Claude Hassoun, Volvic (FR)

Correspondence Address: DOWELL & DOWELL P.C., 103 Oronoco St., Suite 220, Alexandria, VA, 22314, US

(21) Appl. No.: 12/448,815

(22) PCT Filed: Dec. 20, 2007
(86) PCT No.: PCT/FR2007/002134

§ 371 Date: Apr. 16, 2010

(30) Foreign Application Priority Data
Jan. 8, 2007 (FR) 0700078

(51) Int. Cl. A61B 5/03

(52) U.S. Cl. 600/559

(57) ABSTRACT

The invention relates to a non-invasive method for detecting an electric parameter depending on the intralabyrinth pressure in a subject submitted to a repetitive sound stimulation having a predetermined time origin and frequency, by collecting outside the skull of the subject the electric signals emitted by the cochlea in response to said stimulation, wherein said method comprises the following steps: a) sending to the cochlea sound stimulations of the tone burst type with alternating phases; b) collecting the electric responses of the cochlea and the auditive nerve to said stimulations; c) isolating the component of a response corresponding to the average of the electric responses to a positive phase stimulation minus the average of the responses to a negative phase stimulation; d) removing from said isolated response the signals having a time origin identical to the time origin of the stimulation; and e) thus obtaining a cochlea microphonic potential (PMC) of the type representing the intralabyrinth pressure of the subject.

The invention deals with a non-invasive method of measuring an electrical parameter dependent on the intralabyrinthine pressure in a subject. The term “subject” is used here to mean a human being or an animal. The invention also relates to an appliance for implementing such a method.

In the cranial cavity of a subject, the auditory function is handled by organs communicating with the outside. These organs transmit sound vibrations to the nervous system. For this, the sounds are collected in the outer ear, by the antrum auris, and transmitted, via the tympanum, to the organs of the inner ear. These organs are not therefore in direct contact with the outside. They are isolated from the outside by the tympanum and are normally at an internal pressure that corresponds overall to the pressure prevailing inside the cranial cavity.

In the inner ear, the sensory organs responsible on the one hand for the hearing and on the other hand for the balance of the subject intercommunicate and are immersed in liquids. These organs, namely the vestibule for the balance function and the cochlea for the hearing function, together form the labyrinth.

Inside the cochlea, there are internal acoustic hair cells and external acoustic hair cells arranged along a membrane called the basilar membrane.

The basilar membrane vibrates according to the sounds received and separates them according to the frequencies. For this, the membrane has two distinct parts. The part situated close to the middle ear is the thinner and tauter of the two, and collects the high-pitched sounds. The other part is wider and more relaxed, and collects the low-pitched sounds.

The sounds transmitted by the bone system of the ear vibrate the cilia of the acoustic hair cells. These movements modify the characteristics of the medium containing the cells. In particular, the pressure in the cochlea is affected by the vibrations of the cilia. These pressure variations are converted by the organ of Corti into electrical pulses transmitted to the brain by the cochlear nerve. The cochlea therefore operates with respect to the sounds that it receives as a microphone since it “translates” the sounds into electrical pulses. This electrical signal therefore corresponds, indirectly, to the pressure prevailing in the cochlea. The signal is affected by the overall rigidity of the ear. Among other things, the rigidity depends on the intralabyrinthine pressure: the higher the pressure, the more rigid the cochlea is.

The electrical signal emitted by the cochlea is complex. It essentially comprises three types of basic electrical signals.

Firstly, an alternating electrical potential corresponding to the movements of the acoustic hair cells, mainly external, in response to the vibrations of the basilar membrane. This alternating potential, commonly called cochlear microphonic potential (CMP), is directly representative of the sound transmitted to the inner ear, as much with regard to amplitude as with regard to phase and sound frequency.

A second type of basic signal emitted by the cochlea is a continuous electrical potential called summation potential (SP). This summation potential is linked to the movements of the acoustic hair cells, mainly internal. It reproduces the “envelope” of the acoustic stimulation received by the basilar membrane. The amplitude of the continuous potential is all the greater as the frequency of the stimulation increases (high-pitched sounds), whereas the alternating potential is greater in the complex signal at low stimulation frequencies (low-pitched sounds).

A third type of basic signal, called composite action potential (CAP), represents the electrical activity of the neurons and, in particular, of the auditory nerve. It comprises, at the start of the sound stimulation, a negative wave followed by a positive wave, these two waves being overlaid in phase alternation.

In other words, the electrical response of the acoustic hair cells of the cochlea to an acoustic stimulation has three components: a continuous component (SP) reproducing the envelope of the stimulation, an alternating component (CMP), which corresponds to the frequency of the pure sound, and a composite component (CAP) reflecting the activity of the nervous system. This complex electrical response is of the order of a microvolt.

The ratio between the continuous component (SP) and the alternating component (CMP) depends on the frequency of the sound stimulation.

Together, these signals form an overall cochlear response that is usually recorded by an invasive technique called electrocochleography. This technique requires the placement of an electrode as close as possible to the cochlea, that is, it requires the tympanum to be pierced. This technique is therefore difficult to implement and is painful to the subject.

The U.S. Pat. No. 6,589,189 discloses a non-invasive method of indirectly measuring the intracranial pressure based on the acoustic signals emitted by the cochlea. This method is based on the recording of these signals that are reemitted naturally by the cochlea, toward the outer ear, when it is stimulated. These signals, called otoacoustic emissions (OAE), reflect the activity of the external acoustic hair cells. The recording is done using a microphone inserted into the antrum auris to record the response of the cochlea to auditory stimuli.

In this method, it is assumed that the pressure inside the cochlea reflects the intracranial pressure. In other words, it is assumed in U.S. Pat. No. 6,589,189 that the pressure is uniform throughout the cranial cavity. This method is based on the activity of the external acoustic hair cells, provided that the latter are in a condition to operate normally. In other words, these cells must be capable of generating a measurable otoacoustic emission in a reproducible manner on each stimulation and for each ear. This applies as long as there is no deterioration of the hearing in the subject. The absence of otoacoustic emissions participates in the screening, in a non-invasive manner that is known in ENT, of deafness at the level of the cochlea in the subjects.

On the other hand, it is not possible to apply this method when the acoustic signal has disappeared, that is, in most cases where there is an auditory defect. In particular, this method is not applicable to subjects having auditory problems such as Menière's disease.

This disease is reflected in various symptoms that are present simultaneously such as vertigo, tinnitus and deafness to a greater or lesser degree. These symptoms evolve over time, in cycles, the subject being subject to more or less frequent crises. A degradation of the conditions prevailing within the labyrinth, because the hearing and balance organs intercommunicate, induces the appearance of these symptoms.

Menière's disease, in its final state, is reflected in total deafness and the disappearance of the vertigo. This corresponds to a total destruction of the organs involved both in the hearing function and in the balance function.

Various works (Schuknecht H F. Pathology of the ear, 1993. Lea and Febiger, Philadelphia, USA, 672 pp. Kimura R S, Schucknecht H F, Membranous hydrops in the inner ear of the guinea pig after obliteration of the endolymphatic sac. Pract. Otorhinolaryngol. 1965, 27, 343-354. Hallpike C S and Cairns H, Observations on the pathology of Menière's syndrome. Proc. Roy. Soc. Med. 1938, 13, 1317-1331) have shown that this disease could be due to a deregulation of the intralabyrinthine pressure (ILP), with this increasing significantly and no longer being correlated with the intracranial pressure (ICP).

A high intralabyrinthine pressure, associated with Menière's disease, is proven from data collected from autopsies of subjects where a considerable distension of certain intralabyrinthine compartments has been observed. Such a distension indicates an intralabyrinthine hydropy.

This deregulation of the intralabyrinthine pressure could be due, according to certain works (Horner, K. C., Old theme and new reflections: hearing impairment associated with endolymphatic hydrops. Hear. Res. 52 (1991) 147-156. Salt A N, DeMott J. Time course of endolymph volume increase in experimental hydrops measured in vivo with an ionic volume marker. Hear Res. 1994 April; 74 (1-2): 165-72. Portmann M. Meniere's disease, Rev Laryngol Otol Rhinol (Bord). 1990; 111(5): 419-21), to an excess secretion or to an intralabyrinthine liquid resorption defect.

In subjects likely to be affected by Menière's disease, the rapid detection of a variation of the intra-labyrinthine pressure helps to assess a risk of a crisis, and possibly its intensity, which makes it possible to take all measures to avoid the vertigo associated with these crises having unfortunate consequences, for example being the cause of an accident, such as a road accident.

There is therefore an interest in being able to measure a parameter representative of the intralabyrinthine pressure, and in particular detect the variations of this representative parameter in a subject, notably in the case of a subject affected by Menière's disease, which cannot be done with the method described in U.S. Pat. No. 6,589,189.

U.S. Pat. No. 4,741,344 discloses a non-invasive method of collecting electrical signals emitted by the cochlea in response to an auditory stimulation. The collection of the signals is done using an electrode, in a particular configuration, having undergone a surface treatment allowing use with a gel. Such an electrode configuration makes it possible to improve the collection of the signal, given a high background noise. This method, however, does not provide a way of overcoming the artifacts and isolating the response from the cochlea.

U.S. Pat. No. 5,601,091 discloses an audiometric measuring appliance, which is non-invasive and is based on the recording of signals in response to a stimulus. A signal emitted by the brain stem and a signal emitted by the cochlea, in this case an otoacoustic emission (OAE), are recorded. Each signal is collected by a given electrode. The collected signals provide a way of producing the tympanometry and assessing the hearing condition. The presence of background noise or artifacts affects the collection of signals. Moreover, this appliance can be used only to detect the presence of fluid in the middle ear but not measure the intra-labyrinthine pressure.

It is this problem that the invention sets out more particularly to address by proposing a method of detecting a parameter representative of the intra-labyrinthine pressure in a non-invasive, simple and fast manner, in a subject affected by Menière's disease, and an appliance enabling this detection.

To this end, a subject of the invention is a non-invasive method of detecting an electrical parameter dependent on the intralabyrinthine pressure (ILP) in a subject subjected to a repetitive sound stimulation, the time origin and frequency of which are predetermined, by collecting, outside the cranial cavity of the subject, electrical signals emitted by the cochlea in response to this stimulation, this method comprising steps consisting in:

a) sending to the cochlea sound stimulations of the tone burst type with alternating phases,
b) collecting the electrical responses from the cochlea and from the auditory nerve to these stimulations,
c) isolating the component of a response that corresponds to the average of the electrical responses to a positive phase stimulation minus the average of the responses to a negative phase stimulation,
d) eliminating from this isolated response the signals whose time origin is identical to the time origin of the stimulation and
e) thus obtaining a cochlear microphonic potential (CMP) of a type representative of the intralabyrinthine pressure of the subject.

The elimination of the portion of the response comprising the CMP of signals appearing from the start of the stimulation, makes it possible to suppress artifacts and/or interference, in a safe, rapid and total way, from the alternating electrical signal emitted by the cochlea.

There is thus obtained a faithful representation of the propagation of the sound inside the ear. In the event of a variation of the intralabyrinthine pressure, the phase of the sound is affected during its propagation, which is reflected in a variation of the component of the cochlear response comprising the CMP.

Such a method is applicable regardless of the condition of the patient, that is, whether healthy or deaf, since it is independent of the condition of the acoustic hair cells, provided that some still remain. In practice, this method is based on a physical characteristic of the medium, namely the propagation of the sounds, and not on a measurement of a parameter associated with the activity of the medium.

In addition, it provides a way of overcoming artifacts of both biological origin, that is, due to the neurological and/or muscular system, and of external origin, notably those due to the electrical instruments that make up the installation.

According to advantageous but non-mandatory aspects of the invention, such a method can incorporate one or more of the following steps:
it comprises, after the step e), a step of comparison between the response and that obtained in another series of measurements, in order to evaluate a trend of the CMP potentially indicating a change of the ILP.
In the step a), each tone burst of the sound stimulation has a spectrum centered on a frequency of 1 kHz.
In the step a), the stimulation is repeated 20 to 200 times, the time origin being known.
In the step a), a stimulation is effected with phase alternating by 180° every other cycle, so as to eliminate from the response (R₁) the cochlear microphonic potential.
In the step e), the responses appearing before and from two milliseconds are taken into account.

The invention also relates to an appliance for detecting an electrical parameter dependent on the intralabyrinthine pressure to implement a method according to one of the preceding characteristics, comprising a module emitting to the cochlea a repetitive sound stimulation, at least two sensors for an electrical signal emitted by the cochlea in response to the stimulation emitted by the emission module, these sensors being linked to a data collection module, and a control unit, characterized in that the control unit comprises at least one time measuring unit for synchronizing a unit for converting a signal originating from the stimulation emission module with a unit for converting a signal received by the data collection module.

According to advantageous but non-mandatory aspects of the invention, such an appliance can incorporate one or more of the following characteristics:
The stimulation emission module comprises an acoustic tube linking a sound playback device, placed in the vicinity of the antrum auris of the subject, to the stimulation emission module.
The acoustic tube has a known length.
The tube has a minimum length of 30 cm.
It comprises three electrodes, two forming the positive and negative terminals and another forming the ground.
It comprises two time measuring units, one for the stimulation emission module, the other for the response collection module, synchronized with each other.

The invention will be better understood and other benefits of it will become more clearly apparent from reading the following description, given by way of example and with reference to the appended drawings in which:

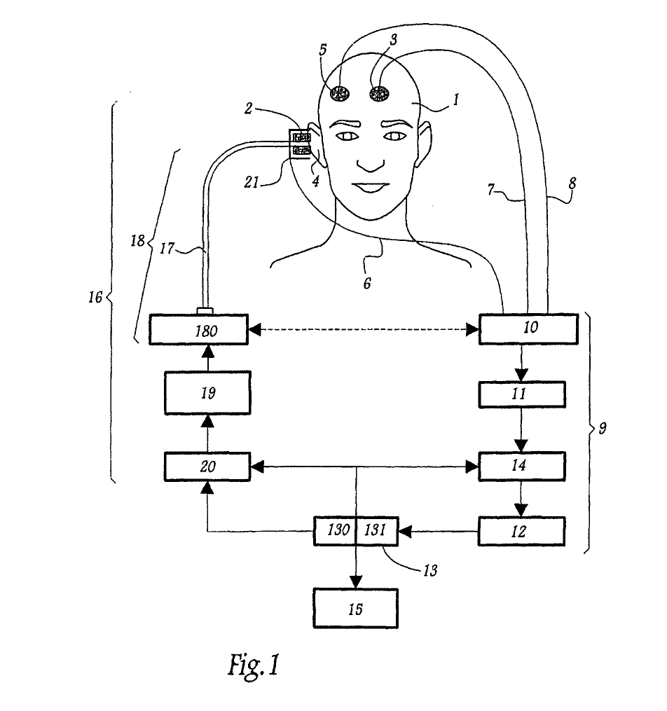

FIG. 1 is a diagrammatic representation of an installation for measuring the intralabyrinthine pressure using the method according to the invention in place on a subject,

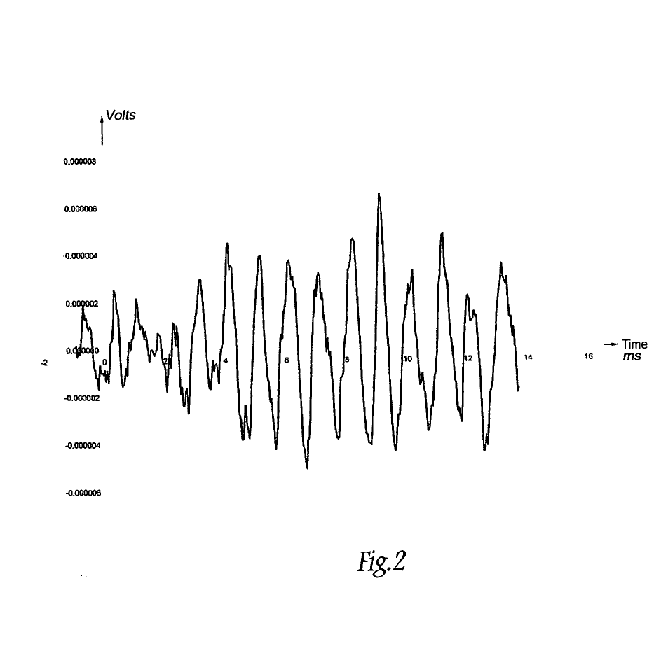

FIG. 2 is a curve illustrating a cochlear microphonic potential, isolated, with the voltage on the y-axis and time on the x-axis, and

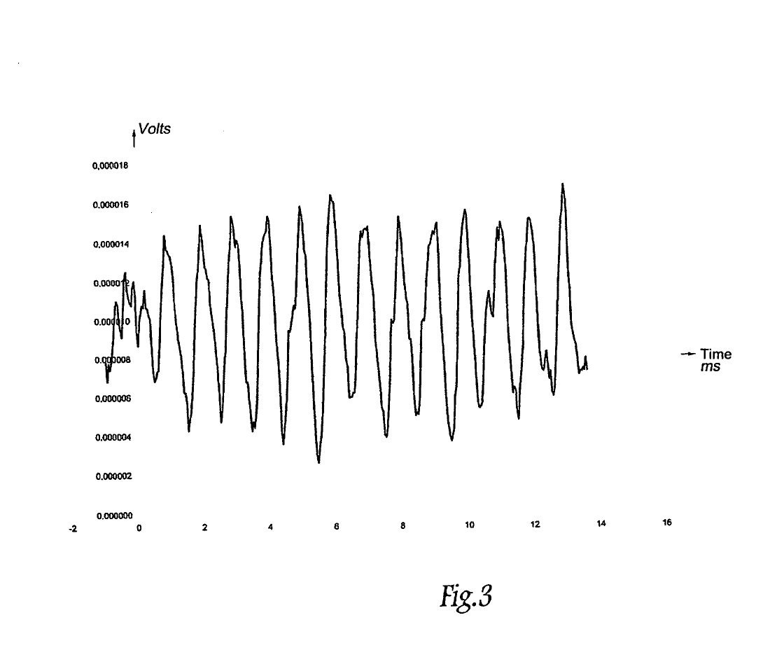

FIG. 3 is a curve similar to FIG. 2 of a non-isolated cochlear microphonic potential, with artifacts.

When a sound stimulation is applied to a subject, the electrical response from the cochlea, denoted R, takes the form of complex signals corresponding to the various cochlea-originated potentials.

This response R corresponds to different electrical potentials.

As a reminder, the composite action potential (CAP) is, generally, the marker of the synchronous activity of the auditory nerve. The summation potential (SP) corresponds to a continuous response from the acoustic hair cells to the stimulation and the microphonic potential (CMP) represents the alternating response emitted by the external acoustic hair cells in response to the stimulation. These parameters are linked by the equation:

R=CAP+SP+CMP.

The microphonic potential (CMP) reproduces the sound stimulation. In other words, knowing that the propagation of a sound depends on the physical/chemical conditions of the medium in which it is propagated, a variation of the CMP is therefore representative of the variation of the physical/chemical conditions prevailing in the sound propagation medium, in the labyrinth. In practice, the organs of the cochlea are immersed, like the other organs of the labyrinth, in a liquid at a given pressure, called intralabyrinthine pressure (ILP).

In a subject with normal hearing, the pressure of the cranial cavity and the intralabyrinthine pressure are roughly the same. On the other hand, for a subject affected by Menière's disease, everything is quite different. In such a subject, a high intralabyrinthine pressure, greater than that of the other regions of the cranial cavity, has been shown (Kimura R S, Schucknecht H F, Membranous hydrops in the inner ear of the guinea pig after obliteration of the endolymphatic sac. Pract. Otorhinolaryngol. 1965, 27, 343-354. Hallpike C S and Cairns H, Observations on the pathology of Meniere's syndrome. Proc. Roy. Soc. Med. 1938, 13, 1317-1331).

One of the difficulties in measuring the intra-labyrinthine pressure in a subject is due to the fact that the various compartments of the labyrinth are included in the temporal bone and are not therefore directly accessible to sensors. It cannot be measured directly and, more importantly, cannot be measured by non-invasive methods.

For an indirect and non-invasive detection of the ILP according to the inventive method, an appliance such as that illustrated in FIG. 1 is used.

This installation comprises sensors for an electrical signal emitted by the cochlea in response to a stimulus, or electrodes, arranged on the head 1 of the subject. At least two sensors 2, 3 are used, one of which, referenced 2, is positioned in the external antrum auris 4, as close as possible to the inner ear but without hurting the subject, notably without piercing the tympanum.

Another sensor 3 is placed on the head 1 of the subject far enough away from the first sensor 2 to record potential differences. Advantageously, this second sensor 3 is placed on the forehead, at a distance of approximately 15 centimeters from the first.

Preferably but in a non-mandatory manner, a third sensor 5 is used, positioned at another point of the head 1 of the subject, for example close to the temple. These three sensors will form three poles for detecting the electrical response R. In this case, the one placed close to the antrum auris forms a positive terminal 2, the one on the forehead a negative terminal 3 and the third sensor 5 forms the ground.

Preferably, electrodes that are known per se such as those used in electrocardiography, are used as sensors. Such electrodes are generally self-adhesive and easy to handle. They can be put in place by the subject himself.

To improve the comfort of the subject, the electrode 2 placed in the antrum auris can be fitted with a foam plug, pierced by a central orifice. Such a foam plug is preferably covered with an anallergenic material, which is itself a good electrical conductor, for example a thin sheet of gold.

These three electrodes 2, 3, 5 are linked by respective wire links 6, 7, 8 to a data collection module 9. Advantageously, the various wires 6, 7, 8 should have a similar length, should not form a loop and/or should not be twisted, in order not to generate electromagnetic disturbances.

This module 9 advantageously comprises a differential preamplification unit 10 for converting differential signals into a signal or referenced response R. In this form, the signal is more easy to manipulate, that is, it can easily be amplified and analog or digital filters can be applied to it using techniques that are known per se.

An amplification unit 11 is positioned downstream of the preamplification unit 10 and upstream of an isolation unit 12.

Inasmuch as the collected signals are of low intensity, since they generally have a voltage of the order of a microvolt, the isolation of the disturbance signal originating from other units of the appliance is advantageously provided by a digital isolation unit 12.

The response R amplified and/or filtered in this way by the amplification unit 11 is directed to a data conversion unit 14, advantageously inserted between the amplification unit 11 and the isolation unit 12. This unit 14 is an analog-digital converter. Such a unit converts the response R into digital data with given resolutions and frequencies.

This analog-digital conversion unit 14 samples the response, of which the start of emission must be perfectly known and synchronized relative to the start of the sound stimulation.

For this, the processing unit 14 is linked to a control unit 13. This control unit 13 comprises a data processing unit 130, for example a microcontroller, and at least one time measuring unit 131.

The time measuring 131 and data processing 130 units are, advantageously, situated so that the measurement of the signal downstream of the unit 12 relative to the amplifier 11 is not disturbed.

As a variant, the data are directed, before or after processing, to a data storage unit 15. This unit 15 comprises media that are known per se, for example digital media such as memory cards, CD-ROMS, DVD-ROMS or analog media, for example paper printouts.

The appliance also comprises a module 16 for stimulating the cochlea of the subject. It should be noted that, to avoid any electromagnetic disturbance, the stimulation module 16 is situated away from the signal collection module 9.

This module 16 comprises an acoustic tube 17 linked, at one end, to the sensor 2 inserted into the ear of the subject and, at the other end, to a loudspeaker 180. Here, the term earpiece 18 designates the loudspeaker 180, the acoustic tube 17 and the sound playback device placed close to the ear of the subject. In the example, the device 21 is an earphone for playing back the sound. One side of this earphone 21 is fitted with a gold leaf forming the electrode 2. In other words, the stimulus playback and response collection devices are one and the same. In an embodiment that is not illustrated, these devices are separate. The earpiece is under the control of an audio amplifier 19 controlled by the control unit 13.

Between the amplifier 19 and the unit 13, a digital-analog conversion unit 20 is inserted, which generates a sound. This data conversion unit 20 converts a digital signal originating from the unit 13 into an analog signal suitable to be amplified by the amplification unit 19.

The analog-digital and digital-analog conversion units 14 and 20 are, under the action of the time measuring unit 131, perfectly synchronized. This way, the time origins of the sound stimulation and of the response R received are known and identical.

Such an appliance is adapted to have a bulk that is small enough to facilitate its transportation and use by the subject, independently. In particular, the energy supply is obtained, according to one aspect of the invention which is not illustrated, by rechargeable batteries or button cells.

This appliance provides a way of applying the non-invasive method of detecting a variation of the intra-labyrinthine pressure, according to the invention and described hereinbelow.

In a first step a), sound stimulations are sent to the cochlea.

The subject, fitted with the sensors 2, 3, 5 as represented in FIG. 1, receives a sound stimulation by the earphone 21 placed close to his external antrum auris 4.

Such a sound playback device 21 should have a high maximum output level, at least of the order of 125 dB, so as to be powered with a low voltage, of the order of to 20 millivolts, for the application envisaged. This makes it possible to obtain an output level of the sound stimulation of the order of 70 to 75 dB above the auditory threshold of the subject, which is compatible with the values that can be accepted by the human ear.

This earphone 21 is linked to the stimulation module 16 by an acoustic tube 17. This tube 17 has a minimum length, so that a time offset arises between the origin of the stimulation, that is, the emission by the stimulation module 16 of a sound, and the playback by the earphone 21 of this sound. This offset, based on the speed of propagation of sound in air, should be of the order of a millisecond, which implies a minimum length for the acoustic tube 17 of approximately 30 centimeters.

This transmission of the sound between the stimulation module 16 and the earphone 21 placed on the ear 4 of the subject is a purely acoustic transmission, using the speed of propagation of sound in air contained in a flexible tube.

The tube 17 does, nevertheless, have a stimulus absorption effect and therefore attenuates the sound perceived at the output of the sound playback device 21. The material, the length and the physical-chemical characteristics of the tube 17 are therefore chosen so as to generate a sound attenuating effect only of the order of 10 dB.

Given the low intensities of the recorded response R, it is important for the mechanical vibration of the earphone 21 placed in the ear of the subject, generally an earphone of auditory prosthesis type, to be minimal. Typically, it is necessary to have a harmonic distortion of the earphone 21 less than 0.5% at the frequencies used, these frequencies being of the order of 1 to 2 kHz.

Similarly, the earphone should exhibit an overall flat response curve for frequencies between 0.5 and 3 kHz.

The sound stimulation emitted by the stimulation module 16 is controlled and known. The sound stimulus sent has a frequency, an amplitude and a phase that are known.

This stimulus is formed from a sinusoidal database. In practice, a sound stimulus of calibrated level is to be produced, capable of reaching 80 to 90 decibels above the normal auditory threshold, for an effective voltage applied to the level of the earphone 21 of a maximum of around one hundred millivolts. Such a voltage minimizes the electrically-originated interfering signals, possibly radiated by the earphone. These interfering signals would in fact be picked up, given their characteristics and their values, by the electrodes 2, 3, 5 positioned on the subject. A stimulus level of 90 decibels provides a way, in subjects having a significant hearing loss, of obtaining a response while observing the maximum that can be tolerated by the ear.

The stimulus, that is, in this case, each sound wave, has a spectrum centered on a frequency of 1 kilohertz. Various works (Avan, P., Büki, B., Maat, B., Dordain, M. and Wit, H. P., Middle-ear influence on oto-acoustic emissions. I: Noninvasive investigation of the human transmission apparatus and comparison with model results, Hear. Res. 140 (2000), 189-201 and Buki B, Chomicki A, Dordain M, Lemaire J J, Wit H P, Chazal J, Avan P. Middle-ear influence on otoacoustic emissions. II: Contributions of posture and intracranial pressure. Hear Res. 2000 February; 140 (1-2): 202-11) have shown that it is at frequencies adjacent to this value that a maximum effect of the intralabyrinthine pressure on the phase of the response emitted by the cochlea is observed.

In another embodiment that is not illustrated, each tone burst has a spectrum centered on a different frequency, for example a frequency of 2 kHz.

Preferably, it is not an isolated sound that is used as the stimulus but a train of stimuli, that is, a train of sound waves whose time origin is exact and determined using the unit 131.

This train of stimuli is produced from sinusoids of known time origin. This origin also serves as time origin for the processing of the response R, that is, of the signals collected by the electrodes 2, 3, 5. For this, the synchronization between the units 20 and 14 must be precise. Advantageously, the units 20 and 14 are subject to the control of one and the same unit 13. In an embodiment that is not illustrated, two time measuring units 131 operating in parallel and synchronized in time are used.

To avoid having the earpiece attacked too abruptly by the train of stimuli, a sound “envelope” is applied to the stimuli. This envelope has a square cosine form of brief duration. It is applied prior to the sending of the stimulating sinusoidal waves which are emitted at full amplitude.

After a certain time that is previously selected by the user, that is, either the subject himself or a third party, the train of stimuli returns to zero, also via a sound envelope in square cosine form, that is symmetrical and opposite to the first. The plateau time, that is, the time where the stimuli are sent, is, overall, between 10 and 80 milliseconds.

This type of stimulation is generally known as a tone burst with alternating positive and negative phases. It in fact involves sending a discontinuous series of waves with a start and a progressive end. In the step a), the stimulation sent is therefore of tone burst type with alternating phases.

It is essential to send such a stimulus rather than a continuous sound stimulation for various reasons. One reason is that, as stated previously, the signal that is to be collected in the response R, in this case the CMP, is very weak and it is generally embedded in a background noise forming an electrical signal that is often ten times greater. In other words, the signal sought, of the order of a microvolt, is embedded in a background noise of the order of one hundredth of a millivolt.

This general background noise comprises a component of biological origin, that is, originating from the nervous and/or muscular system. This component is a noise that is random and non-repetitive by definition. In other words, it cannot be reproduced and its intensity is not known. Its intensity is still, however, moderate compared to the CMP, although generally greater than the CMP. This biological component is independent of the stimulus. It is revealed in FIGS. 2 and 3 where an electrical signal is recorded before the start of the stimulation. This is the portion of the curves situated to the left of the y axis.

If this stimulus is repeated identically, that is, a series of tone bursts is sent several times in succession, typically between 20 and 200 times, tone bursts are emitted that are always identical with regular and therefore easily identifiable intervals.

In a second step b), the electrical responses R from the cochlea and from the auditory nerve to these stimulations are collected. The processing of the responses by averaging, synchronous with the emission of the train of stimuli, makes it possible to differentiate and extract, from the complex response received, a signal representative of the stimulation received, that is, to isolate the cochlear microphonic potential (CMP). In other words, in a third step c), the component of a response that corresponds to the average of the electrical responses to a positive stimulation minus the average of the responses to a negative stimulation is isolated.

The CMP is a response representative of the stimulus. In other words, by thus sending a train of stimuli in the form of tone bursts whose origin is known, it is known that the CMP will be emitted proportionally to the stimulus, at known and regular instants. Thus, it is easy to identify relative to the background noise of biological origin.

Nevertheless, there is a second interfering component of the background noise. This second component is sufficiently strong to mask the CMP. This component is not random and generally perfectly known. It is the electrical noise transmitted by the earpiece 18 itself, in fact a radiated electrical interfering signal.

This second noise has a form similar to that of the emitted stimulus. In other words, this interfering noise is repetitive and it is emitted with an intensity and a frequency that are quite comparable to that of the CMP since it is also linked to the stimulus. It is therefore, a priori, difficult to be able to extract the CMP alone, without the noise generated by the installation.

For this, for a long time, the CMP recordings have been considered to be of little interest and unreliable since they were easily interfered with by the measuring instruments, the interfering noise being sufficiently strong to mask the CMP, with the additional risk of being taken for the CMP by an insufficiently skilled operator.

Because of this, in this type of recording, it is known to deliberately eliminate the interfering noise and the CMP from the response collected by providing a stimulation with opposite phases. Once every two times, the stimulus is sent with a phase φ a first time and a phase φ+π the time after. Thus, the signal received is cancelled out and, if the interfering signal is well suppressed, the CMP is also suppressed. In this case, the response R₁ comprises no more than the summation potential SP and the composite action potential CAP according to the equation

R₁=SP+CAP

In other words, from the difference between the response R received and the response R₁ received with a stimulation with phase alternating by 180° once every two times, an overall value for the cochlear microphonic potential CMP can be deduced. This CMP must then be “cleaned” of any artifact and interference of electrical origin.

In a fourth step d), the signals whose time origin is identical to the time origin of the stimulation are eliminated from this isolated response.

For this, the fact that the radiated interfering signal is of electromagnetic origin, unlike the CMP which is of acoustic origin, is used.

This interfering signal is in fact transmitted by the earpiece 18 linked to the stimulation module 16 as soon as the stimulation module 16 is switched on.

This interfering signal therefore appears in the response R collected from the start of the emission of the stimulus, since it is emitted directly by the electrical appliances of the installation as soon as they are switched on.

In other words, the speed of propagation of the interfering signal is not comparable to the speed of propagation of the signal emitted by the cochlea. In one case, that of the interfering signal, the propagation is that of an electrical wave, which is to say it is almost instantaneous, and in the other case, that of the signal emitted by the cochlea, it is the propagation of a sound in air then in the organism that is to be taken into account.

In other words, if there is an acoustic tube 17 of approximately 30 centimeters in length, the propagation time of the sound between the module 16 and the earpiece 21 is approximately one millisecond. This sound received by the sound playback device 21 is emitted by the latter, passes into the ear of the subject and stimulates the acoustic hair cells which vibrate and respond to this sound in the form of an electrical signal (CMP+SP) which is then detected by the electrodes 2, 3, 5.

Works (Zwislocki J J. Some current concepts of cochlear mechanics. Audiology. 1983; 22(6): 517-29) have shown that the phase shift between the stimulation emitted by the earpiece 21 and its reception by the cochlea, called go phase shift, results from the time taken for the sound to be propagated through the structure of the ear, which is known to be approximately one millisecond (Békésy G. von. Direct observation of the vibrations of the cochlear partition under a microscope. Acta Otolaryngol. 1952 June; 42(3): 197-201).

In other words, between the instant when the stimulation is emitted by the stimulation module 16 and the instant when the electrical response emitted by the cochlea is collected via the electrodes 2, 3 and 5, approximately 2 milliseconds elapse.

The interference of electromagnetic origin is radiated immediately by the loudspeaker 180, that is, before the first millisecond, and it is detected by the electrodes almost simultaneously.

The electrical oscillation emitted in response to the stimulus by the cochlea, that is, the CMP, can be detected only a minimum of two milliseconds after the start of the stimulation and in no case can there be an emission of an oscillation from the cochlea before the first millisecond. In practice, before this first millisecond, the cochlea cannot have received stimulation, the stimulus still being propagated in the tube 17 upstream of the earpiece 21.

Given the length of the tube 17 and the speed of propagation of the sound in air, it is in fact impossible for this stimulation to be transmitted by the earpiece 21 before one millisecond.

Any response received during this first millisecond can therefore only have an origin other than the cochlea, that is, be due to an interference of an electro-magnetic kind. A response R received during these first two milliseconds reflects interference of radiated electrical origin. FIG. 3 illustrates a response of this type. The peak-to-peak amplitude of the response reaches 10 microvolts, from the first millisecond, which roughly corresponds to the maximum value of the response.

In other words, the total absence of response R around the frequency corresponding to that of the stimulus, generally adjacent to 1 kHz, during the first two milliseconds, guarantees that there is no radiated electrical interference.

In order to quantify this interfering signal, a stimulation is given with a frequency that varies between two values. The propagation of the stimulus induces a phase variation on the signal received according to the frequency. The interfering signal of electrical origin has a fixed phase between these two frequencies. The CMP has a different phase according to the frequency of the stimulation. The variation of the phase with the frequency is roughly equal to 2π.τ, where τ is the signal propagation delay. For the interfering signal for which τ is zero, the propagation being immediate, the phase does not depend on the frequency. Such a stimulation with variable frequency is known by the name of “stimulation sweep” or “chirp”. This is no more and no less than a sound stimulation with the frequency of the sinusoid modulated.

In this way, the artifact is quantified and extracted from the overall CMP in order to obtain only the CMP emitted by the cochlea. FIG. 2 illustrates a response of this type. The maximum of the response is approximately 0.6 microvolt, a response around 0.4 microvolt being recorded only after 4 milliseconds after the start of the stimulation. It will be noted that, up to 2 milliseconds, the intensity recorded corresponds to the intensity recorded before the stimulation, that is, the recording of a biological background noise. The signal representative of the response from the cochlea reaches its full amplitude only from approximately 3 milliseconds after the start of the stimulation.

The typical CMP, without interference, emitted by the cochlea, corresponds to the transmission of sound in the ear, that is, through a given medium. If the medium in which the sound is propagated is subject to a pressure variation, the phase is affected on propagation and therefore the CMP, which is an electrical parameter, is itself also affected. The typical CMP therefore reflects a value of the intralabyrinthine pressure ILP.

In a fifth step e), the duly obtained response is considered to be a cochlear microphonic potential of the type representative of the ILP.

Tests carried out with animals, with a direct, invasive measurement of the pressure in parallel with the non-invasive measurement of the CMP according to the method described here, have shown that a variation, however rapid, of the ILP can be detected just as rapidly, by comparing the CMPs between two series of measurements. It should be noted that, for one and the same patient, the same measurement conditions normally give the same indications relating to the ILP. There is therefore reproducibility in the method. Since these CMPs are stripped of all interfering signals in the manner described previously, they are representative of the ILP and it is possible to appreciate whether a change of the ILP has taken place, in a sixth step of comparing the responses obtained in two series of measurements.

This indirect detection of the ILP is implemented for a subject affected by Menière's disease regardless of the stage of the disease, provided that the subject is not profoundly deaf. In this case, it is the final stage of the disease, and the subject no longer has vertigo or hearing.

Such a method, which is easy to implement, simple and rapid, enables the subject to regularly detect the trend of an electrical parameter dependent on the ILP. Any change of this parameter potentially indicates a change of ILP. This change is a factor to be taken into account in detecting the appearance of vertigo and the prevention of its consequences.

Such a method, provided the subject is not deaf, can be applied to a healthy subject or at least one not affected by Menière's disease.

1.-10. (canceled)

11. A non-invasive method of detecting an electrical parameter dependent on the intralabyrinthine pressure (ILP) in a subject subjected to a repetitive sound stimulation, the time origin and frequency of which are predetermined, by collecting, outside the cranial cavity of the subject, electrical signals emitted by the cochlea in response to this stimulation, this method comprising steps consisting in:
a) sending to the cochlear sound stimulations of the tone burst type with alternating phases,
b) collecting the electrical responses (R) from the cochlea and from the auditory nerve to these stimulations,
c) isolating the component of a response that corresponds to the average of the electrical responses to a positive phase stimulation minus the average of the responses to a negative phase stimulation,
d) eliminating from this isolated response the signals whose time origin is identical to the time origin of the stimulation and
e) thus obtaining a cochlear microphonic potential (CMP) of a type representative of the intralabyrinthine pressure of the subject.

12. The method as claimed in claim 11, characterized in that it comprises, after the step e), a step of comparison between the response and that obtained in another series of measurements, in order to evaluate a trend of the CMP potentially indicating a change of the ILP.

13. The method as claimed in claim 11, characterized in that, in the step a), each tone burst of said sound stimulation has a spectrum centered on a frequency of 1 kHz.

14. The method as claimed in claim 11, characterized in that, in the step a), the stimulation is repeated 20 to 200 times, the time origin being known.

15. The method as claimed in claim 11, characterized in that, in the step a), a stimulation is effected with phase alternating by 180° every other cycle, so as to eliminate from the response (R) the cochlear microphonic potential (CMP) and to obtain a response (R1) containing only interfering signals (SP, CAP) thus identified, which can be eliminated, in subsequent steps, from the response (R) in order to isolate an overall cochlear microphonic potential (CMP).

16. The method as claimed in claim 11, characterized in that, in the step e), the responses appearing before and from two milliseconds are taken into account.

17. An appliance for detecting an electrical parameter dependent on the intralabyrinthine pressure to implement a method as claimed in claim 11, comprising a module emitting to the cochlea a repetitive sound stimulation, at least two sensors for an electrical signal emitted by the cochlea in response to the stimulation emitted by the emission module, said sensors being linked to a data collection module, a data processing unit, a data storage unit, a control unit comprising at least one time measuring unit for synchronizing a unit for converting a signal originating from the stimulation emission module with a unit for converting a signal received by the data collection module, characterized in that the stimulation emission module comprises an acoustic tube linking a sound playback device, placed in the vicinity of the antrum auris of the subject, to the stimulation emission module and in that the acoustic tube has a known length.

18. The appliance as claimed in claim 17, characterized in that said tube has a minimum length of 30 cm.

19. The appliance as claimed in claim 17, characterized in that it comprises three electrodes, two forming the positive and negative terminals and another forming the ground.

20. The appliance as claimed in claim 17, characterized in that it comprises two time measuring units, one for the stimulation emission module, the other for the response collection module, synchronized with each other.
